(19) United States
(12) Patent Application Publication
(10) Pub. No.: US 2006/0234919 A1
(43) Pub. Date: Oct. 19, 2006

(54) Use

(76) Inventors: Kerstin Uvnäs-Moberg, DJURSHOLM (SE); Thomas Lundeberg, Lidingö (SE)

Correspondence Address: YOUNG & THOMPSON, 745 SOUTH 23RD STREET, 2ND FLOOR, ARLINGTON, VA, 22202, US

(21) Appl. No.: 10/488,166

(22) PCT Filed: Sep. 2, 2002
(86) PCT No.: PCT/SE02/01560

§ 371 Date: Mar. 1, 2004

(30) Foreign Application Priority Data
Aug. 31, 2001 (SE) 0102910-7

(51) Int. Cl. A61K 38/22

(52) U.S. Cl. 514/9

(57) ABSTRACT

The present invention relates to the use of substances with oxytocin for the preparation of pharmaceutical composition against inflamation. It also relates to a pharmaceutical composition comprising at least one substance with oxytocin activity against inflamation.

The present invention relates to the use of substances with oxytocin activity for the preparation of a pharmaceutical composition against inflammation. It also relates to a pharmaceutical composition comprising at least one substance with oxytocin activity against inflammation.

### BACKGROUND OF THE INVENTION

Oxytocin was one of the first peptide hormones to be isolated and sequenced. It is a nonapeptide with two cysteine residues that form a disulphide bridge between positions 1 and 6 and corresponds to the formula

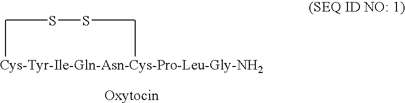

In the human body oxytocin is produced in the paraventricular nucleus, PVN, and the supraoptic nucleus, SON, of the hypothalamus. It differs by only two amino acids from vasopressin, which is also produced in these nuclei. The magnocellular oxytocinergic neurones of the SON and PVN send oxons to the posterior pituitary from which oxytocin is released into the circulation. Parvocellular neurones that originate in the PVN project into multiple areas within CNS. The oxytocin-producing cells are innervated by cholinergic, catecholaminergic as well as peptidergic neurones. The presence of oxytocin in different tissues outside the brain, such as the uterus, ovaries, testis, thymus, adrenal medulla and pancreas has been demonstrated and oxytocin is suggested to exert local effects in these organs.

A parallel secretion of oxytocin into the brain regions and into the circulation occurs in response to some stimuli such as suckling, but other stimuli can cause separate activation of oxytocinergic neurones, terminating in the brain or the pituitary.

For a long time the only effects attributed to oxytocin were its stimulating effects on milk ejection and uterine contractions, but in the past decades it has been shown that oxytocin exerts a wide spectrum of effects within the central nervous system, CNS. It has been suggested that oxytocin participates in the control of memory and learning processes and of various types of behaviour such as feeding, locomotion, as well as maternal and sexual behaviour. Oxytocin is also suggested to participate in the control of cardiovascular functions, thermoregulation, and pain threshold and fluid balance. There is also evidence that oxytocin is involved in the control of various immunological processes. It has recently been demonstrated that oxytocin injections cause a lowering of blood pressure and increased weight gain—long lasting effects after repetitive administration. As a central stimulating substance oxytocin plays an important role in the interaction between mother and progeny in mammals. The products may also be used prophylactic in young human beings e.g. already in new born babies or young children to prevent the development of diseases later on in life which diseases are dependent on stress conditions during the fetal life. Such conditions may be heart/vessel diseases such as stroke, heart infarct, hypertension, and diabetes.

It has now turned out that oxytocin has a relieving effect on inflammation.

There are several oxytocin derivatives, i.e. compounds with a structure similar to that of oxytocin. The inventors have preliminary indications that other oxytocin derivatives than oxytocin may give the effects against inflammation, as well as parts of the oxytocin molecule. Such oxytocin derivatives and parts of the oxytocin molecule with the same or similar effects against inflammation as oxytocin are generally called substances with oxytocin activity. Substances with oxytocin activity also include precursors, metabolic derivatives, oxytocin agonists and analogues displaying the same properties.

The oxytocin like substances may be used in all kinds of inflammation conditions. It has turned out that they may be used especially advantageously against edema (Examples 1-4), hyperalgesia (Example 5), myeloperoxidase accumulation (Example 6), cystitis (Examples 7, 9, and 10), pancreatitis (Example 8), cutaneous inflammation (Example 11), allergic rhinitis (Example 12), dermatitis (Example 13), air-way inflammations (Example 14), and asthma (Example 15).

### PRIOR ART

Abstract of RU 2145870 discloses that oxytocin may be used in order to treat patients with diabetes mellitus complicated with suppurative inflammation diseases.

Abstract of SU 1528502 discloses that oxytocin may be used in combination with antibiotics in order to treat pyorrhoea inflammatory cases.

Abstract of RU 2157206 discloses that oxytocin may be used in order to treat chronic suppurative middle otitis and trepanation cavity inflammation.

Rev. Obst. Gin. Venezuela, 1968, 28, p. 169-172 shows that oxytocin in aerosol form may be used in order to treat mammal engorgement.

However, the above-mentioned prior art documents do not disclose that oxytocin like substances may be used against edema, hyperalgesia, myeloperoxidase accumulation, cystitis, pancreatitis, cutaneous inflammation, allergic rhinitis, dermatitis, air-way inflammation, and asthma.

### SUMMARY OF THE INVENTION

The present invention relates to the use of a substance with oxytocin activity for the preparation of a pharmaceutical composition against inflammation. The invention also relates to a pharmaceutical composition comprising an effective concentration of at least one substance with oxytocin activity in mixture or otherwise together with at least one pharmaceutically acceptable carrier or excipient. Such a pharmaceutical composition could be used in order to achieve a relieving effect on inflammation.

The effect of oxytocin can be extended or strengthened by administration in combination with drugs increasing the release of oxytocin and/or the number or the affinity of oxytocin receptors. One such drug is oestrogen. The effect of oxytocin can also be extended or strengthened by administration in combination with drugs having an α₂-agonistic effect. One such drug is clonidine.

### DETAILED DESCRIPTION OF THE INVENTION

One object of the present invention is the use of a substance with oxytocin activity for the preparation of a pharmaceutical composition against inflammation.

Examples of inflammation according to the present invention are edema, hyperalgesia, myeloperoxidase accumulation, cystitis, pancreatitis, cutaneous inflammation, allergic rhinitis, dermatitis, air-way inflammation, and asthma.

It is preferred that the substance is selected from the group consisting of the following compounds: 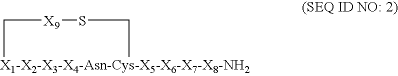
wherein
X₁ is selected from the group consisting of Cys, Mpa and nothing,
X₂ is selected from the group consisting of Tyr, (O-methyl-Tyr), Phe, and nothing,
X₃ is selected from the group consisting of Ile, Val, Hoph, Phe, Cha, and nothing,
X₄ is selected from the group consisting of Gin, Ser, Thr, Cit, Arg, and Daba,
X₅ is selected from the group consisting of Pro, and nothing,
X₆ is selected from the group consisting of Ile, Leu, nothing, Val, Hos, Daba, Thr, Arg, and Cit,
X₇ is selected from the group consisting of Gly, nothing, and Ala,
X₈ is selected from the group consisting of Gly, and nothing,
X₉ is selected from the group consisting of CH₂ and S;
as well as salts thereof.

The cystein disulfide bridge is only present when X₁ represents Cys or Mpa, X₂ represents Tyr, (O-methyl-Tyr) or Phe, and X₃ represents Ile, Val, Hoph, Phe or Cha.

By “nothing” is meant that the letters respectively may have no meaning or may represent a bond and that there may be a direct bond between the items (letter, atom or group) situated to the right and to the left, respectively, of the letter designating “nothing”. For example, in SEQ ID NO: 2 above, when only X₁ designates nothing, the resulting molecule corresponds to X₂-X₃-X₄-Asn-Cys-X₅-X₆-X₇-X₈-NH₂. When only X₈ designates nothing, the X₇ residue is amidated.

It is even more preferred that the substance is selected from the group consisting of the following compounds: 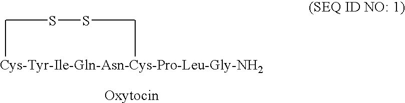
X₁ is Cys, X₂ is Tyr, X₃ is Ile, X₄ is Gln, X₅ is Pro, X₆ is Leu, X₇ is Gly, X₈ is nothing, and X₉ is S in Claim 3 and 8 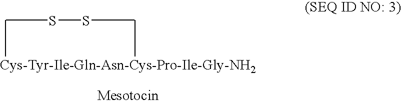
X₁ is Cys, X₂ is Tyr, X₃ is Ile, X₄ is Gln, X₅ is Pro, X₆ is Ile, X₇ is Gly, X₈ is nothing, and X₉ is S in Claim 3 and 8 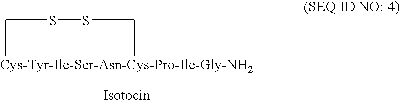
X₁ is Cys, X₂ is Tyr, X₃ is Ile, X₄ is Ser, X₅ is Pro, X₆ is Ile, X₇ is Gly, X₈ is nothing, and X₉ is S in Claim 3 and 8 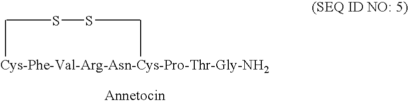
X₁ is Cys, X₂ is Phe, X₃ is Val, X₄ is Arg, X₅ is Pro, X₆ is Thr, X₇ is Gly, X₈ is nothing, and X₉ is S in Claim 3 and 8 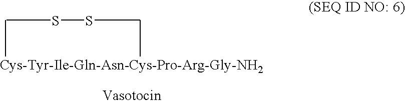
X₁ is Cys, X₂ is Tyr, X₃ is Ile, X₄ is Gin, X₅ is Pro, X₆ is Arg, X₇ is Gly, X₈ is nothing, and X₉ is S in Claim 3 and 8 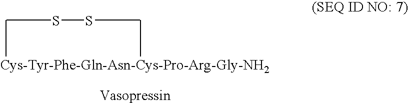
X₁ is Cys, X₂ is Tyr, X₃ is Phe, X₄ is Gln, X₅ is Pro, X₆ is Arg, X₇ is Gly, X₈ is nothing, and X₉ is S in Claim 3 and 8 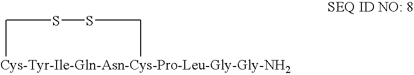
X₁ is Cys, X₂ is Tyr, X₃ is Ile, X₄ is Gln, X₅ is Pro, X₆ is Leu, X₇ is Gly, X₈ is Gly, and X₉ is S in Claim 3 and 8 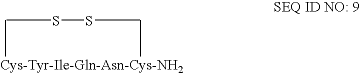
X₁ is Cys, X₂ is Tyr, X₃ is Ile, X₄ is Gln, X₅-X₈ is nothing, and X₉ is S in Claim 3 and 8 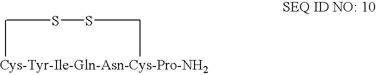
X₁ is Cys, X₂ is Tyr, X₃ is Ile, X₄ is Gln, X₅ is Pro, X₆-X₈ is nothing, and X₉ is S in Claim 3 and 8

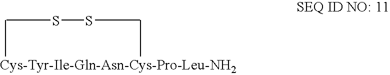

X₁ is Cys, X₂ is Tyr, X₃ is Ile, X₄ is Gln, X₅ is Pro, X₆ is Leu, X₇-X₈ is nothing, and X₉ is S in Claim 3 and 8

| | |
|---|---|
| Tyr-Ile-Gln-Asn-Cys-Pro-Leu-Gly-NH₂ | SEQ ID NO: 12 |

X₁ is nothing, X₂ is Tyr, X₃ is Ile, X₄ is Gln, X₅ is Pro, X₆ is Leu, X₇ is Gly, X₈ is nothing, and X₉ is S in Claim 3 and 8

| | |
|---|---|
| Ile-Gln-Asn-Cys-Pro-Leu-Gly-NH₂ | SEQ ID NO: 13 |

X₁-X₂ is nothing, X₃ is Ile, X₄ is Gln, X₅ is Pro, X₆ is Leu, X₇ is Gly, X₈ is noting, and X₉ is S in Claim 3 and 8

| | |
|---|---|
| Gln-Asn-Cys-Pro-Leu-Gly-NH₂ | SEQ ID NO: 14 |

X₁-X₃ is nothing, X₄ is Gln, X₅ is Pro, X₆ is Leu, X₇ is Gly, X₈ is nothing, and X₉ is S in Claim 3 and 8

| | |
|---|---|
| Ile-Gln-Asn-Cys-Pro-NH₂ | SEQ ID NO: 15 |

X₁-X₂ is nothing, X₃ is Ile, X₄ is Gln, X₅ is Pro, X₆-X₈ is nothing, and X₉ is S in Claim 3 and 8 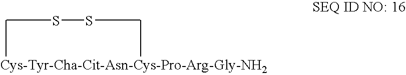
X₁ is Cys, X₂ is Tyr, X₃ is Cha, X₄ is Cit, X₅ is Pro, X₆ is Arg, X₇ is Gly, X₈ is nothing, and X₉ is S in Claim 3 and 8 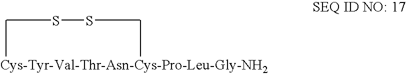
X₁ is Cys, X₂ is Tyr, X₃ is Val, X₄ is Thr, Xs is Pro, X₆ is Leu, X₇ is Gly, X₈ is nothing, and X₉ is S in Claim 3 and 8 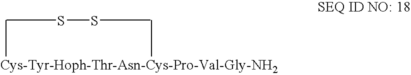
X₁ is Cys, X₂ is Tyr, X₃ is Hoph, X₄ is Thr, X₅ is Pro, X₆ is Val, X₇ is Gly, X₈ is nothing, and X₉ is S in Claim 3 and 8 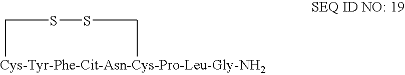
X₁ is Cys, X₂ is Tyr, X₃ is Phe, X₄ is Cit, X₅ is Pro, X₆ is Leu, X₇ is Gly, X₈ is nothing, and X₉ is S in Claim 3 and 8 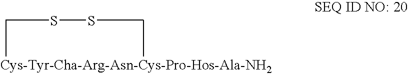
X₁ is Cys, X₂ is Tyr, X₃ is Cha, X₄ is Arg, X₅ is Pro, X₆ is Hos, X₇ is Ala, X₈ is nothing, and X₉ is S in Claim 3 and 8 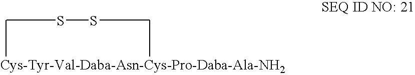
X₁ is Cys, X₂ is Tyr, X₃ is Val, X₄ is Daba, X₅ is Pro, X₆ is Daba, X₇ is Ala, X₈ is nothing, and X₉ is S in Claim 3 and 8 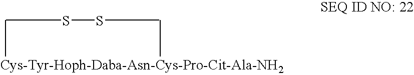
X₁ is Cys, X₂ is Tyr, X₃ is Hoph, X₄ is Daba, X₅ is Pro, X₆ is Cit, X₇ is Ala, X₈ is nothing, and X₉ is S in Claim 3 and 8 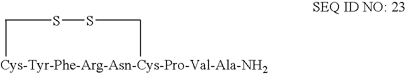
X₁ is Cys, X₂ is Tyr, X₃ is Phe, X₄ is Arg, X₅ is Pro, X₆ is Val, X₇ is Ala, X₈ is nothing, and X₉ is S in Claim 3 and 8 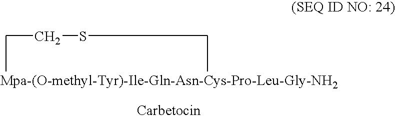
X₁ is Mpa, X₂ is (O-methyl-Tyr), X₃ is Ile, X₄ is Gln, X₅ is Pro, X₆ is Leu, X₇ is Gly, X₈ is nothing, and X₉ is CH₂ in Claim 3 and 8,
wherein Mpa stands for β-mercaptopropionic acid; wherein the CH₂—S-group thereof is bonded to the cystein portion via a thioether bond i position 6 giving the structure for SEQ ID NO: 24 as follows: 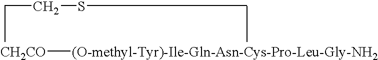
wherein (O-methyl-Tyr) stands for O-methyltyrosine of the chemical formula: 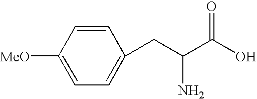
wherein Cha stands for cyclohexylalanine, 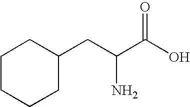
Hoph stands for homophenylalanine, 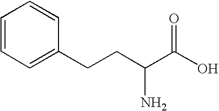
Cit stands for citrulline, 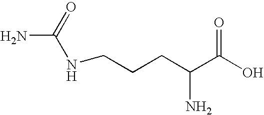
Daba stands for diaminobutyric acid, and 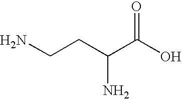
Hos stands for homoserine.

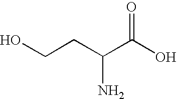

There are different processes described for the synthetical production of oxytocin; commercial processes are for instance described in U.S. Pat. Nos. 2,938,891 and 3,076,797.

A substance with oxytocin activity refers, whenever applicable, in addition to oxytocin also to precursors, metabolic derivatives, oxytocin agonists or analogues displaying the same properties.

Annetocin has been isolated from the earthworm, as described in Oumi T, Ukena K, Matsushima O, Ikeda T, Fujita T, Minakata H, Nomoto K, Annetocin: an oxytocin-related peptide isolated from the earthworm, Eisenia foetida, Biochem Biophys Res Commun 1994, January 14; 198(1): 393-399. The uterotonic activity and myometrial receptor affinity of carbetocin is described in Atke A and Vilhardt H, Acta Endocrinologica (Copenh) 1987, 115: 155-160.

Other substances with oxytocin activity could also be used, such as naturally occurring or artificially modified variants, analogues, and derivatives of oxytocin, mesotocin, isotocin, and annetocin. Such substances could be obtained by addition, insertion, elimination, or substitution of at least one amino acid in these hormones. By a substance with an oxytocin like activity is also understood precursors, metabolites such as metabolic derivatives e.g. metabolic degradation products, agonists, or analogues of the substances mentioned herein displaying the same properties. When one or more amino acids are added to a substance with oxytocin activity, it is preferred to add 1-3 amino acids to the carboxyl terminal. Metabolic derivatives or metabolic degradation products may be oxytocin like peptides e.g. with nine amino acids such as oxytocin, mesotocin, isotocin, and annetocin from which one or more amino acids has been deleted from either the carboxyl terminal end or the amino terminal end or both the carboxyl terminal and the amino terminal end, preferably 1-3 amino acids from each terminal. It could be ascertained that these variants are analogues of oxytocin, mesotocin, isotocin or annetocin by immunological methods, e.g. RIA (radioimmunoassay), IRMA (radiometric methods), RIST (radioimmunosorbent test), and RAST (radioallergosorbent test). The invention also includes substances having at least 50, 60, 70, 80 and most preferably 90% homology to oxytocin, and showing oxytocin activity.

As mentioned above, there are indications that addition of one or more amino acids to the oxytocin molecule may give a molecule that has effects against inflammation. One example of such a molecule is SEQ ID NO: 8.

As mentioned above, there are indications that subfragments of the oxytocin molecule have effects against inflammation. Examples of subfragments of the oxytocin molecule are the following compounds: SEQ ID NO: 9, SEQ ID NO: 10, SEQ ID NO: 11, SEQ ID NO: 12, SEQ ID NO: 13, SEQ ID NO: 14, and SEQ ID NO: 15.

There is also a possibility to create new compounds with oxytocin activity by means of computer simulation. Methods for computer simulation are known by a person skilled in the art, e.g. as described in EP 0660 210 A2. Eight new compounds have been created by means of computer simulation, namely the following peptides: SEQ ID NO: 16, SEQ ID NO: 17, SEQ ID NO: 18, SEQ ID NO: 19, SEQ ID NO: 20, SEQ ID NO: 21, SEQ ID NO: 22, and SEQ ID NO: 23.

The invention also relates to the peptides mentioned above in both D- and L-form. Especially the invention relates to the L-form. By inversion of the peptide sequence thereof, the D-form could be converted to the L-form. The effect of the D- and L-forms are the same. These and the peptides above can be produced by methods known to a person skilled in the art, e.g. according to Merrifield, P. B., “Solid Phase Synthesis”, Angew. Chemie, 1985, No. 97, p. 801.

It is preferred that a substance with oxytocin activity is administered in an amount of 0.01-100 ng/kg body weight of the patient, in particular 0.1-10 ng/kg.

Another object of the invention is a pharmaceutical composition against inflammation comprising an effective concentration of at least one substance with oxytocin activity in mixture or otherwise together with at least one pharmaceutically acceptable carrier or excipient. It is preferred that the substance is selected from the group consisting of compounds with the formula SEQ ID NO: 2. Examples of inflammation according to the present invention edema, hyperalgesia, myeloperoxidase accumulation, cystitis, pancreatitis, cutaneous inflammation, allergic rhinitis, dermatitis, air-way inflammation, and asthma. It is even more preferred that the substance is selected from the group consisting of the following compounds: SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, and SEQ ID NO: 24.

The pharmaceutical compositions according to the invention may contain substances that extend or strengthen the effects of oxytocin. Such substances could increase the release of oxytocin and/or the number or affinity of oxytocin receptors, such as oestrogen, or drugs having an α₂-agonistic effect, such as clonidine.

It should be noted that salts of the compounds according to the invention are included within the scope of the invention. As examples of salts of the compounds are intended in particular pharmaceutically acceptable acid and base addition salts.

The expression “pharmaceutically acceptable acid addition salts” are intended to be any non-toxic organic or inorganic acid addition salt of the compounds of SEQ ID NO: 2. Examples of illustrative inorganic acids that form suitable salts are hydrochloric acid, hydrobromic acid, sulphuric acid, phosphoric acid and acid metal salts such as sodium monohydrogen ortophosphate and potassium hydrogensulphate. Examples of illustrative organic acids that form suitable salts are mono-, di- and tricarboxylic acids. Examples of such acids are acetic acid, glycolic acid, lactic acid, pyruvic acid, malonic acid, succinic acid, glutaric acid, fumaric acid, malic acid, tartaric acid, citric acid, ascorbic acid, maleic acid, hydroxymaleic acid, benzoic acid, hydroxybenzoic acid, phenylacetic acid, cinnamic acid, salicylic acid, 2-phenoxybenzoic acid, and sulphonic acids such as p-toluenesulphonic acid, methanesulphonic acid and 2-hydroxyethanesulphonic acid. Such salts could either be in hydrated or anhydrous form. The acid addition salts of these compounds are generally water soluble and different hydrophilic organic solvents and, that compared to the free base forms thereof, generally display higher melting points.

The expression “pharmaceutically acceptable base addition salts” are intended to be any non-toxic organic or inorganic base addition salt of the compounds of SEQ ID NO: 2. Examples of illustrative inorganic bases that form suitable salts are alkali and earth alkali metal hydroxides and carbonates such as sodium hydroxide, sodium carbonate, potassium hydroxide, potassium carbonate, calcium hydroxide, calcium carbonate, magnesium hydroxide, magnesium carbonate and ammonia. Examples of illustrative organic bases that form suitable salts are methylamine, dimethylamine, trimethylamine and picoline. Either mono- or dibasic salts could be formed with such compounds. The base addition salts of these compounds are generally water soluble and different hydrophilic organic solvents and, that compared to the free base forms thereof, generally display higher melting points.

The pharmaceutical compositions are prepared in a manner known to a person skilled in the pharmaceutical art. The carrier or the excipient could be a solid, semisolid or liquid material that could serve as a vehicle or medium for the active ingredient. Suitable carriers or excipients are known in the art. The pharmaceutical composition could be adapted to oral, parenteral, intravaginal, or topical use and could be administered to the patient as tablets, capsules, suppositories, solutions, suspensions or the like.

The pharmaceutical compositions could be administered orally, e.g. with an inert diluent or with an edible carrier. They could be enclosed in gelatine capsules or be compressed to tablets. For oral therapeutic administration the compounds according to the invention could be incorporated with excipients and used as tablets, lozenges, capsules, elixirs, suspensions, syrups, wafers, chewing gums and the like. These preparations should contain at least 4% by weight of the compounds according to the invention, the active ingredient, but could be varied according to the special form and could, suitably, be 4-70% by weight of the unit. The amount of the active ingredient that is contained in compositions is so high that a unit dosage form suitable for administration is obtained.

The tablets, pills, capsules, lozenges and the like could also contain at least one of the following adjuvants: binders such as microcrystalline cellulose, gum tragacanth or gelatine, excipients such as starch or lactose, disintegrating agents such as alginic acid, Primogel, corn starch, and the like, lubricants such as magnesium stearate or Sterotex, glidants such as colloidal silica dioxide, and sweetening agents such as saccharose or saccharin could be added or flavourings such as peppermint, methyl salicylate or orange flavouring. When the unit dosage form is a capsule it could contain in addition to the type above a liquid carrier such as polyethylene glycol or a fatty oil. Other unit dosage forms could contain other different materials that modify the physical form of the unit dosage form, e.g. as coatings. Accordingly, tablets or pills could be coated with sugar, shellac or other enteric coating agents. A syrup could in addition to the active ingredient contain saccharose as a sweetening agent and some preservatives, dyes and flavouring agents. Materials that are used for preparation of these different compositions should be pharmaceutically pure and non-toxic in the amounts used.

For parenteral administration the compounds according to the invention could be incorporated in a solution or suspension. Parenteral administration refers to the administration not through the alimentary canal but rather by injection through some other route, as subcutaneous, intramuscular, intraorbital, intracapsular, intraspinal, intrasternal, intravenous, intanasal, intrapulmonary, through the urinary tract, through eye drops, rectal or intravaginal (e.g. as a suppository, a vagitorium, a cream or an ointment), through the lactiferous tract in cattle, into an organ such as bone marrow, etc. Bone marrow may also be treated in vitro. These preparations could contain at least 0.1% by weight of an active compound according to the invention but could be varied to be approximately 0.1-50% thereof by weight. The amount of the active ingredient that is contained in such compositions is so high that a suitable dosage is obtained.

The solutions or suspensions could also comprise at least one of the following adjuvants: sterile diluents such as water for injection, saline, fixed oils, polyethylene glycols, glycerol, propylene glycol or other synthetic solvents, antibacterial agents such as benzyl alcohol or methyl paraben, antioxidants such as ascorbic acid or sodium bisulfite, chelating agents such as ethylene diamine tetraacetic acid, buffers such as acetates, citrates or phosphates, and agents for adjustment of the tonicity such as sodium chloride or dextrose. The parenteral preparation could be enclosed in ampoules, disposable syringes or multiple dosage vessels made of glass or plastic.

For topical administration the compounds according to the invention could be incorporated in a solution, suspension, or ointment. These preparations could contain at least 0.1% by weight of an active compound according to the invention but could be varied to be approximately 0.1-50% thereof by weight. The amount of the active ingredient that is contained in such compositions is so high that a suitable dosage is obtained. The administration could be facilitated by applying touch, pressure, massage, heat, warms, or infrared light on the skin, which leads to enhanced skin permeability. Hirvonen, J., Kalia, Y N, and Guy, R H. Transdermal delivery of peptides by iontophoresis, Nat Biotechnol 1996 December; 14(13): 1710-1713 describes how to enhance the transport of a drug via the skin using the driving force of an applied electric field. Preferably, iontophoresis is effected at a slightly basic pH.

Other administration forms are inhalation through the lungs, buccal administration via the mouth, enteral administration via the small intestine, and local administration with a release, preferably a slow release, of the active substance e g in the form of a ring. All these administration forms could be effected by means known by a person skilled in the art.

Oxytocin may be used in any type of inflammation conditions. The inventors have shown that oxytocin administered s.c. reduced experimentally induced inflammation, as measured as the volume of the carrageenan induced edema and the myeloperoxidase activity in the rat paw (Example 1).

Since glucocorticoids are potent anti-inflammatory agents the effect of dexamethasone on the carrageenan induced inflammation as a complement to the oxytocin experiments was examined (Example 4). As expected, dexamethasone reduced the carrageenan-induced edema significantly. Oxytocin, when administered in high doses, may increase corticosterone levels acutely in rats, and therefore it could be argued that the anti-inflammatory effect of oxytocin was caused by a rise in corticosterone. However, the effect of oxytocin was equally potent as the effect of dexamethasone, and also the lower dose of oxytocin (100 μg/kg s.c.), which does not induce a similar increase in corticosterone levels as 1000 μg/kg of oxytocin, had a strong inhibitory effect on inflammation. Additionally, also oxytocin administered i.c.v. may increase corticosterone levels acutely in rats. This was tested in Example 3 and did not decrease the paw edema. The oxytocin administered i.c.v. should have decreased inflammation if the effect was mediated through an increase in corticosterone levels. Therefore, it is not likely that oxytocin caused its anti-inflammatory effect through a rise in corticosterone only, although corticosterone could of course have participated in the effect.

Another reason for examining the effect of oxytocin i.c.v. on the experimentally induced inflammation was that the inventors had previously have shown that oxytocin at 1 μg/kg i.c.v. increases the survival of ischaemic musculocutaneous skin flaps in rats. Oxytocin at 1 mg/kg s.c. induced the same effect, which was abolished by the oxytocin antagonist (Example 2) at the same dose (1 mg/kg s.c.). In other studies they have shown that this oxytocin antagonist in this dose also counteract the effects of oxytocin on for example weight gain, feeding and nociceptive thresholds in rats.

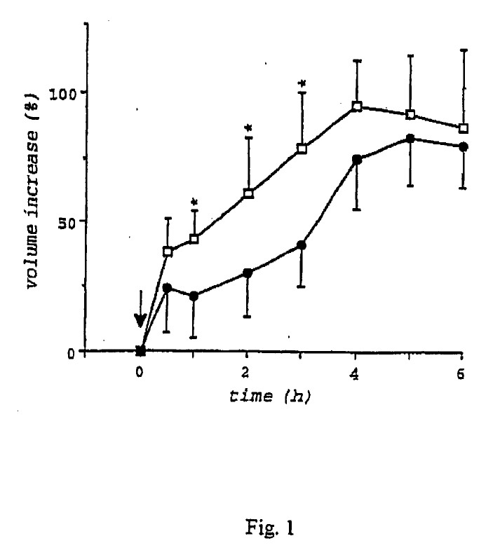

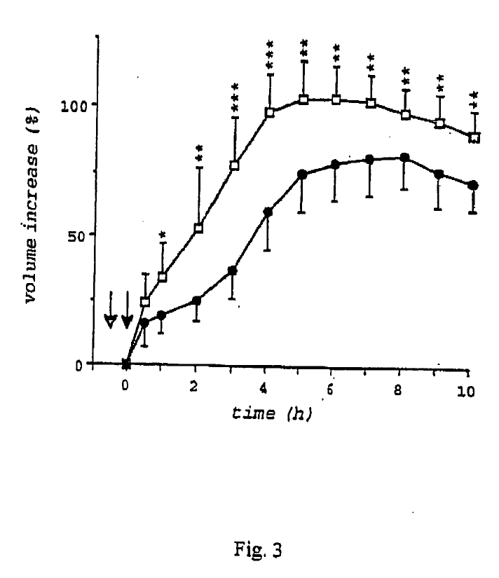

In contrast, some of the effects of oxytocin on blood pressure are not counteracted by this antagonist, nor does it replace oxytocin receptor binding in the rat heart. This oxytocin antagonist, as also some other oxytocin antagonists, has been found to have agonistic properties in some experimental models, which might explain the even stronger effect of oxytocin 1 mg/kg s.c. administered together with the antagonist shown in FIG. 3 (when compared to FIG. 1). However, this difference might also be just a normal variance between the experiments, since the activity of myeloperoxidase was significantly lower when measured 6 hours after the injection of oxytocin 1 mg/kg s.c. alone and since oxytocin at a 10-fold lower dose induced a similar long-lasting effect as oxytocin and the oxytocin antagonist administered together. Carrageenan is also known to induce hyperalgesia, and earlier studies have found that oxytocin has an antinociceptive effect which is most prominent 30-60 minutes after the injection. In Example 5, it was found that oxytocin-treated rats had significantly higher nociceptive thresholds both at 1 and 6 hours after the injection of oxytocin. Thus oxytocin seems to have diminished the carrageenan induced hyperalgesia.

Since neutrophil recruitment is one important mediator of inflammation, and the enzyme myeloperoxidase, which is abundant in neutrophil leukocytes, has been found to be a reliable marker for the detection of neutrophil accumulation in inflamed skin in vivo, the activity of myeloperoxidase in the rat paw injected with carrageenan was measured (Example 6). Oxytocin reduced the neutrophil content, as measured as myeloperoxidase activity, in the carrageenan induced inflammation significantly.

In conclusion, this study showed that oxytocin may have anti-inflammatory effects in vivo, an effect of oxytocin that to our knowledge not previously has been shown. Such an effect of oxytocin would be physiologically suitable to protect females against inflammation during parturition and breastfeeding, which are periods when oxytocin is released in high amounts.

Interstitial cystitis (IC) was first described by Hunner in 1915 as a painful bladder condition manifested by urinary frequency, nocturia, urgency, suprapubic pain and ulcers on the vesical mucosa revealed by cystoscopy. The classical Hunner's ulcers are rarely seen, but bleeding on refilling the bladder after cystoscopic distension is common. It is estimated that 0.5 million people have IC in the USA and that 90% of patients with IC are female. Although IC is a common disease the treatment is largely empirical, because the cause of the disease is unknown. Afferent c-fibres, characterised by high sensitivity to the neurotoxin capsaicin, are involved in the pathogenesis of bladder hyper-reflexia and detrusor instability. Because oxytocin is like sensory stimulation is effective in treating visceral pain and urinary dysfunction, oxytocin might be effective for treating urinary frequency in patients with IC. In Example 7 the effect of oxytocin instillation on IC was evaluated in a rat model. Oxytocin instillation reduces urinary frequency in rats with hydrochloric acid-induced cystitis.

In Examples 8-11, the effects of the oxytocin have been investigated in 4 experimental models in vivo which involve the endogenous release of quinines and are thought to resemble various severe acute inflammatory diseases. In summary, oxytocin was shown to be effective in inhibiting symptoms mediated by the endogenous release of quinines in experimental models of visceral and cutaneous inflammation.

Allergic rhinitis, one of the most common allergic diseases, is accompanied by rhinorrhea, sneezing, pruritus and congestion. These symptoms are considered to becaused by antigen-antibody reaction on mast cells that are located on the epithelia of the nasal cavity. Activation of mast cells results in the release of numerous chemical mediators including histamine, leukotrienes, prostaglandins, platelet-activating factor (PAF) and cytokines that can in turn recruit additional inflammatory cells, trigger the release of further inflammatory mediators and stimulate afferent nerves. It is well known that nasal spraying with corticosteroid is a therapy for several types of chronic rhinitis. Topical application of oxytocin on allergic rhinitis was studied in Example 12.

This is the first report that nasal symptoms induced by antigen-antibody reaction in rats are inhibited by oxytocin suggesting that it may inhibit sneezing and nasal pruritus in allergic rhinitis patients.

The magnesium deficiency-induced dermatosis in hairless rats is a useful animal model for the exploration of potential treatments for cutaneous inflammatory disorders. In Example 13, topical administration of oxytocin was tested. It prevents and inhibits atopic dermatitis-like symptoms in hypomagnesaemic hairless rats.

Cytokines have been suggested to play key roles in the pathogenesis of bronchial asthma, which is an inflammatory disease, dominated by eosinophilic granulocytes. In Example 14, the effect of oxytocin on the production of TNF-alpha in eosinophilic airway inflammation in rats was investigated. TNF-alpha increased significantly 3 h postchallenge. Treatment with the oxytocin effectively inhibited the increase in the TNF-alpha concentration.

One of the most effective anti-inflammatory treatments available for asthma is glucocorticoid therapy. This is likely to be due to multiple effects on the inflammatory response, including reduced production of cytokines by lymphocytes (Barnes P J. Anti-inflammatory therapy for asthma. Annu Rev Med 1993, 44: 229-242) and reduced expression of adhesion molecules, such as intercellular adhesion molecule-1, by vascular endothelial cells and airway cells. In vitro, oxytocin may decrease the release of some interleukins. Therefore, the effect of oxcytocin on OVA-induced N-acetyl-LTE4 synthesis in BN rats—an experimental asthma model—was tested in Example 15. Inhaled oxytocin inhibits OVA-induced N-acetyl-LTE4 synthesis in BN rats—an experimental asthma model.

It is evident that oxytocin may be used against any type of inflammation.

All publications mentioned herein are hereby incorporated by reference. By the expression “comprising” we understand including but not limited to. Thus, other non-mentioned substances, additives or carriers may be present.

The invention will be illustrated by the following Examples, which are only intended to illustrate and not restrict the invention in any way.

The invention will now be described with reference to the following Figures of which

FIG. 1 shows the percentage increase from pre-treatment values in hindpaw volume in rats treated s.c. with NaCl (□) (n=8) or oxytocin (1.0 mg/kg) (●) (n=8) immediately before induction of acute inflammation by means of a s.c. carrageenan injection (indicated by the filled arrow) into the hindpaw. The results are shown as means±SD. Statistical evaluation was performed by means of a one-way ANOVA, followed by Fisher's test for post-hoc comparisons. *p<0.05, compared to controls. F(1,84)=5.72, p=0.031

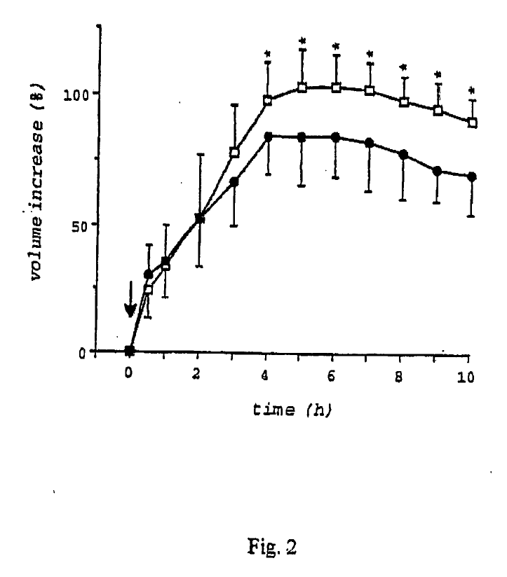

FIG. 2 shows the percentage increase in hindpaw volume in rats treated s.c. with NaCl (□) (n=8) or oxytocin (0.1 mg/kg) (●) (n=8) right before induction of acute inflammation by means of a s.c. carrageenan injection (indicated by the filled arrow) into the hindpaw. The results are shown as means±SD. Statistical evaluation was performed by means of a one-way ANOVA, followed by Fisher's test for post-hoc comparisons. *p<0.05, compared to controls. F(1,140)=5.09, p=0.041

FIG. 3 shows the percentage increase in hindpaw volume in rats treated s.c. with NaCl (□) (n=8) or oxytocin (1.0 mg/kg)+the oxytocin antagonist (1.0 mg/kg) (●) (n=8) right before induction of acute inflammation by means of a s.c. carrageenan injection (indicated by the filled arrow, oxytocin antagonist administration is indicated by the unfilled arrow) into the hindpaw. The results are shown as means±SD. Statistical evaluation was performed by means of a one-way ANOVA, followed by Fisher's test for post-hoc comparisons. *p<0.05, **p<0.01 and ***p<0.001, compared to controls. F(1,140)=20.82, p=0.0004

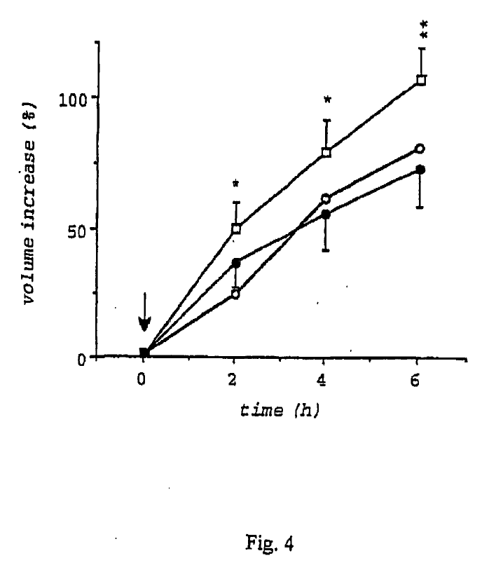

FIG. 4 shows the percentage in hindpaw volume in rats treated i.m. with NaCl (□) (n=6) or dexamethasone (10 mg/kg) (●) (n=6) right before induction of acute inflammation by means of a s.c. carrageenan injection (indicated by the filled arrow) into the hindpaw. The results are shown as means±SD. The oxytocin-treated rats from FIG. 3 are shown for comparison (◯). Statistical evaluation was performed by means of a one-way ANOVA, followed by Fisher's test for post-hoc comparisons. *p<0.05, **p<0.01, dexamethasone compared to saline-treated controls. F(1,20)=18.6, p=0.0015

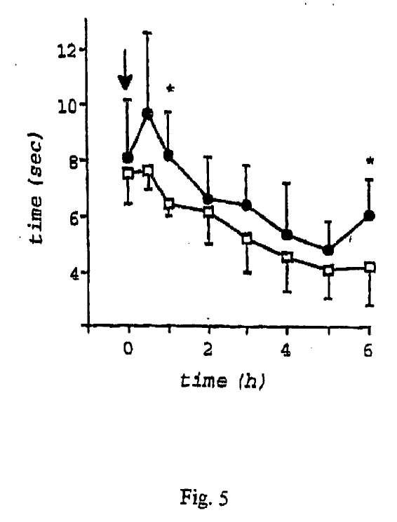

FIG. 5 shows the nociceptive thresholds in response to the Randall Selitto Test in rats treated s.c. with NaCl (□) (n=8) or oxytocin (1.0 mg/kg) (●) (n=8) immediately before induction of acute inflammation by means of a s.c. carrageenan injection (indicated by the filled arrow) into the hindpaw. The results are shown as means±SD. Statistical evaluation was performed by means of a one-way ANOVA, followed by Fisher's test for post-hoc comparisons. *p<0.05, compared to controls. F(1,84)=6.68, p=0.022

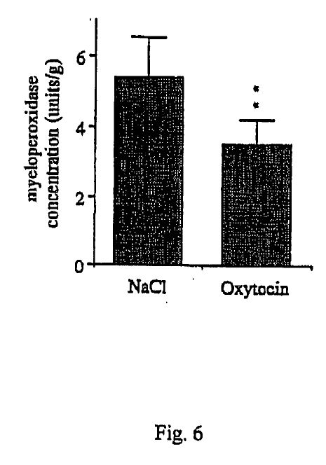

FIG. 6 shows the accumulation of myeloperoxidase in the right hindpaw of rats 6 hours after s.c. treatment with NaCl (n=6) or oxytocin (1.0 mg/kg) (n=6) right before induction of acute inflammation by means of a s.c. carrageenan injection into the hindpaw. The results are shown as means±SD. Statistical evaluation was performed by a Student's t-test. **p<0.01.

### EXAMPLE 1-5

### Effect of Oxytocin on Carrageenan Induced Inflammation

The following materials were used in these Examples.

Animals

Male Sprague-Dawley rats (260-300 g for s.c. injected and 340-380 g for i.c.v. injected) were used (B&K Universal A B, Sollentuna, Sweden). The animals arrived at least one week before experiments and were housed three-four per cage (except animals provided with i.c.v. cannulas that were housed individually) with free access to food (R36, Ewos, Södertälje, Sweden) and water. The light schedule was a 12/12 h light/dark cycle, and ambient temperature was 20±2° C.

Drugs

Oxytocin and the oxytocin antagonist (1-deamino-2-D-Tyr-(OEt)-4-Thr-8-Orn-oxytocin) (Ferring, Malmö, Sweden) were dissolved in physiological saline and injected in a volume of 1 ml/kg s.c. in the dorsal neck. Dexamethasone (Decadron®, Merck, Sharp & Dome, USA) was administered intramuscularly (i.m.). Oxytocin given i.c.v. was dissolved in a volume of 5 μl physiological saline and slowly injected over a period of 1 minute through the i.c.v. guide cannula via a 25 G stain-less-steel injection needle connected to a 10 μl Hamilton syringe via a polyethylene tube. Controls received saline in the same amounts.

### EXAMPLE 1

### The Effects of Oxytocin s.c. on Carrageenan Induced Edema

The model of inflammation used, the carrageenan induced edema, is a commonly used model for studies of inflammation and for testing novel anti-inflammatory drugs (Winter C A, Risley E A, Nuss G W. Carrageenan-induced edema in hind paw of the rat as an assay for anti-inflammatory drugs. Proc Soc Exp Biol Med 1962; 111:544-7). To produce acute inflammation, carrageenan, 2 mg in 0.1 ml saline, was injected s.c. into the plantar region of the rat right hindpaw.

The rats were treated s.c. with oxytocin (1.0, 10, 100 or 1000 μg/kg (n=8 in each group) or saline (n=8+8) immediately before the carrageenan injection. The edema of the right hindpaw was measured (volume in ml) using a plethysmometer (Ugo Basile, type 7150, Florence, Italy) before treatment, half an hour after, one hour after, and then every hour up to 6 or 10 hours after treatment.

The results are presented as means±SD. Statistical analysis was performed by means of a 1-way ANOVA, followed by Fisher's test for post-hoc comparisons. In the analysis of myeloperoxidase accumulation, a Student's t-test was used. P-values of 0.05 or less were regarded as statistically significant.

Oxytocin (1000 μg/kg s.c.) decreased the carrageenan induced edema significantly at 1-3 hours after the injection compared to saline injected controls (1 hour: 21±16 % vs. 43±11%; p<0.05, 2 hours: 30±17% vs. 61±22%; p<0.05 and 3 hours: 41 ±16% vs. 79±21%; p<0.05) (values calculated as percentage increase from pre-treatment values) (ANOVA; F(1,84)=5.72, p=0.031) (FIG. 1).

A tenfold lower dose of oxytocin (100 μg/kg s.c.) reduced the edema significantly 4-10 hours after the injection (ANOVA; F(1,140)=5.09, p=0.041) (FIG. 2). No effect was observed in response to oxytocin 1.0 and 10 μg/kg s.c. (data not shown).

### EXAMPLE 2

### Effect of Oxytocin Antagonist on the Anti-Inflammation Effect of Oxytocin on Carrageenan Induced Edema

The rats were treated s.c. with oxytocin (1000 μg/kg) and the oxytocin antagonist (1000 μg/kg) (the antagonist was given 30 minutes before oxytocin) (n=8), the oxytocin antagonist alone (1000 μg/kg) (n=8) or saline (n=8) immediately before the carrageenan injection. Measurements as in Example 1.

The oxytocin antagonist (1000 μg/kg s.c.) did not abolish the oxytocin (1000 μg/kg s.c.) induced effect on carrageenan induced edema. In this experiment, a significant reduction of the hindpaw volume compared to controls was seen 1-10 hours after the injection of carrageenan (ANOVA; F(1,140)=20.82, p=0.0004) (FIG. 3). The significant difference was gone when measured at 24 hours (data not shown). The oxytocin antagonist (1000 μg/kg s.c.) administered alone did not induce any effect (data not shown).

### EXAMPLE 3

### The Effects of Oxytocin i.c.v. on Carrageenan Induced Edema

The rats were treated i.c.v. with oxytocin (1.0 μg/kg) (n=6) or saline (n=7) immediately before the carrageenan injection. Measurements as in Example 1. The animals were anaesthetised with sodiumpentobarbital (50 mg/kg) (Apoteksbolaget, Sweden) injected intraperitoneally (i.p.). The scull was uncovered, a hole was drilled in the right parietal bone and a guide cannula (21 G) was fixed stereotactically to the scull by means of acrylic dental cement. The coordinates were 1.00 mm posterior and 1.30 mm lateral to the bregma. The guides reached but did not penetrate the dura mater. The injection needles (25 G) reached 3.80 mm below the dura mater, with the tip of the needle in the right lateral ventricle. The animals were allowed one week of recovery after the operation. At the end of the experiment, the placement of the guide cannula was checked by injection of 2 μl of toluidine blue.

Oxytocin administered i.c.v (1.0 μg/kg) did not decrease the paw edema (ANOVA; F(1,110)=2.73, p=0.13) (data not shown).

### EXAMPLE 4

### The Effects of Dexamethasone i.m. on Carrageenan Induced Edema

Since glucocorticoids are potent anti-inflammatory agents we examined the effect of dexamethasone on the carrageenan induced inflammation as a complement to the oxytocin experiments.

The rats were treated i.m. with dexamethasone (10 mg/kg) (n=6) or saline (n=6) immediately before the carrageenan injection. The edema of the right hindpaw was measured using the plethysmometer before treatment, and at 2, 4 and 6 hours after treatment.

Dexamethasone (10 mg/kg i.m.) decreased the carrageenan induced edema significantly when measured at 2, 4 and 6 hours after the injection (2 hours: 35±9.3% vs. 48±10%; p<0.05, 4 hours: 54±14% vs. 78±12%; p<0.05 and 6 hours: 71±15% vs. 105±11%; p<0.01) (FIG. 4).

### EXAMPLE 5

### The Effects of Oxytocin s.c. on Carrageenan Induced Hyperalgesia

Carrageenan is also known to induce hyperalgesia (Satoh M, Kuraishi Y, Kawamura M. Effects of intrathecal antibodies to substance P, calcitonin-gene related peptide and galanin on repeated cold stress-induced hyperalgesia: comparison with carrageenan-induced hyperalgesia. Pain 1992; 49:273-8), and earlier studies have found that oxytocin has an antinociceptive effect which is most prominent 30-60 minutes after the injection (Lundeberg T, Uvnäs-Moberg K, Ågren G, Bruzelius G. Anti-nociceptive effects of oxytocin in rats and mice. Neurosci Lett 1994; 170:153-7). Nociceptive thresholds were measured in the rats given oxytocin 1000 μg/kg s.c. and their controls. This was done by determining the response to mechanical stimulation using the Randall Selitto Test (Ugo Basile, type 7200, Italy). The mechanical stimulus was applied to the dorsal surface of the hindpaw by a wedged-shape pusher at a loading rate of 48 g/s and the pressure required to initiate the struggle response was measured. All rats were trained on three consecutive days before testing.

Carrageenan decreased nociceptive thresholds significantly (ANOVA; F(1,84)=22.6, p=0.0001). Oxytocin treated rats (1000 μg/kg s.c.) responded significantly different to the mechanical stimulation (ANOVA; F(1,84)=6.68, p=0.022) and showed an increase in withdrawal latency of the paw at 1 hour and 6 hours after the injection, compared to controls (p<0.05) (FIG. 5).

### EXAMPLE 6

### The Effects of Oxytocin s.c. on Carrageenan Induced Myeloperoxidase Accumulation

The rats were treated s.c. with oxytocin (1000 μg/kg) (n=6) or saline (n=6) immediately before the carrageenan injection. The accumulation of myeloperoxidase in the rat right hindpaw was measured 6 hours after the treatment with oxytocin or saline, and carrageenan. To determine the recruitment of neutrophils in response to the carrageenan induced inflammation in the rat hindpaw, the paws were weighed and homogenised in 10 ml 0.5% hexadecyltrimethyl-ammonium bromide (Sigma Chemical Co, USA), and freeze-thawed, whereafter the myeloperoxidase activity of the supernatant was assessed. The enzyme activity was determined spectrophotometrically as the change in absorbance at 650 nm (25° C.) occuring in the redox reaction of H₂O₂-tetramethylbenzidine (Sigma Chemical Co, USA) catalysed by myeloperoxidase. Values are expressed as myeloperoxidase units/g tissue. Oxytocin 1000 μg/kg s.c. reduced the neutrophil content, as measured as myeloperoxidase activity, in the carrageenan induced inflammation significantly. The myeloperoxidase concentration (i.e. the neutrophil recruitment) in the hindpaw was 3.5±0.72 units/g tissue in the oxytocin-treated rats compared to 5.4±1.1 units/g in the saline-treated controls (p<0.01) (FIG. 6).

### EXAMPLE 7

### The Effect of Oxytocin Instillation on Urinary Frequency in Rats with Hydrochloric Acid-Induced Cystitis

Materials and Methods

Twenty Sprague-Dawley rats (body weight 220-250 g) were randomly divided into four equal groups: group 1, normal controls; group 2, a sham treatment (HCl-induced IC, with subsequent saline instillation); group 3, a sham treatment (HCl-induced IC, with subsequent acetic acid instillation); and group 4, stimulated (HCl-induced IC with oxytocin and acetic acid instillation). The IC animal model developed by Rivas et al. was used (Rivas D A, Chancellor M B, Shupp-Byme D, Shenot P J, McHugh K, McCue P. A molecular marker for the development of interstitial cystitis in a rat model: isoactin gene expression. J. Urol. 1997; 157: 1937-1940); IC was induced in groups 2-4 under general anaesthesia (using a combined subcutaneous injection with acepromazine 0.05 mg/kg, ketamine 50 mg/kg and xylazine 5 mg/kg). A sterile polyethylene catheter (outside diameter 1.27 mm; Becton Dickinson Company, Sparks, Md., USA) was inserted into the bladder through the urethra and all urine aspirated. HCl diluted in saline (0.4 mol/l, 0.5 ml) was slowly instilled into the bladder with a sterile syringe. A prophylactic antibiotic (Bactrim, 2.2 mg/kg) was administered 1 h before urethral catheterization and 5 ml of saline injected intraperitoneally daily to hydrate the rats during the first 3 days after intravesical HCl instillation to prevent blood clots forming in the urine. None of the 20 rats died during the experiment. All chemicals were obtained from Sigma-Aldrich unless otherwise indicated.

The micturition pattern was measured both before and 3 weeks after intravesical HCl instillation, using a Grass 79 Polygraph (Grass Instruments Co., Quincy, Mass., USA). Rats were placed into a metabolic cage and micturition recorded automatically for 17 h (17.00-09.00 hours); all rats were assessed at the same time of the day to minimise the effect of the circadian cycle on micturition frequency and volume. The rats were allowed free access to water during the overnight assessment. Rat food was not provided during the recording of micturition because it might have influenced the recording of micturition.

Each rat in group 4 was administered with oxytocin 1 mg/ml for 10 days every third day for 3 weeks. The animals showed no obvious discomfort. All the procedures were identical in groups 2-4 except for the oxytocin administration in group 4.

Three weeks after HCl treatment the rats were catheterised through the urethra under general anaesthesia. Mineral oil was applied to the skin and mucosa adjacent to the urethral orifice to prevent chemical irritation caused by acetic acid. The bladder was evacuated through the urethral catheter, followed by acetic acid instillation (0.75%, 0.5 ml) every 15 min for 2 h, to prevent acetic acid dilution by urine in the bladder.

Results

To analyse the micturition frequency groups 2 and 3 were combined because they were identical except for the intravesical instillation with acetic acid on the day of death. The micturition frequency before intravesical instillation with HCl was not significantly different among the groups (1, 2+3 and 4), at 10.7 (2.8), 12.1 (2.3) and 11.2 (2.7) voids/17 h, respectively. All rats in groups 2+3 and 4 had mild gross haematuria during the first 3 days after intravesical instillation with HCl. Three weeks after inducing IC the micturition frequency in groups 2+3 was twice that in the controls (P=0.01). After 3 weeks, in group 4 the micturition frequency was significantly less than in group 2+3 (P=0.05). The urine volume per micturition in group 4 was not significantly higher than in group 2+3. Thus, oxytocin instillation reduces urinary frequency in rats with hydrochloric acid-induced cystitis.

### EXAMPLE 8-11

### Effects of Oxytocin in Models of Visceral and Cutaneous Inflammation

### EXAMPLE 8

### Caerulein-Induced Pancreatitis

Caerulein-induced pancreatitis: Female Sprague-Dawley rats (200-250 g) were anaesthetized with pentobarbitone sodium and phenobarbitone sodium (each 40 mg/kg, i.p.). The cholecystokinin analogue caerulein (8 nmol/kg in 2 h) was infused via one jugular vein for exocrine hyperstimulation and induction of acute edematous pancreatitis (Lampel M, Kern H F. Acute interstitial pancreatitis in the rat in-duced by excessive doses of a pancreatic secretagogue. Virchows Arch (Pathol Anat) 1997; 373: 97-117), while control animals were infused with a corresponding volume (4 ml/kg/h) of phosphate-buffered saline. Pre-treatment with the kininase II inhibitor captopril (50 m mol/kg, i. p.) was given 30 min prior to the infusion of caerulein. At the end of the experiment, the animals were sacrificed with an overdose of pentobarbital sodium. The pancreatic tissue was dried for 16 h in a vacuum centrifuge and the edema was quantified as fluid wt. per dry wt. of the tissue. For the determination of lipase activity in serum, the blood was collected following decapitation in separate groups of rats. The pancreas of these animals was excised an placed immediately into 2 ml of a 154 mmol/l solution of NaCl. After centrifugation at 2×10⁵ m/s² for 25 min the supernatant was stored at −80° C. All samples were assayed for lipase activity (lipase test Boehringer Mannheim, Germany).

Results

The i. v. infusion of caerulein (4 nmol/kg/h for 2 h) induced acute edematous pancreatitis demonstrated by an increase in water content of the pancreatic tissue from 3.12±0.52 g/g d. wt. (n=10) to 8.11±0.96 g/g d. wt. (n=16). Subcutaneous pre-treatment with oxytocin (1 mg/ml) caused a significant inhibition of the edema formation to 5.25±0.68 g/g d. wt. (n=16). The effect of oxytocin on the activities of lipase in the blood serum and in the pancreatic tissue was investigated. Small amounts of active lipase (0.24±0.26 U/mg; n=10) could be detected in the pancreatic tissue of control animals. During caerulein-induced pancreatitis this value was increased more than 10-fold to 4.87±0.72 U/mg (n=10). This increase was significantly attenuated by 1 mg/ml s.c. of oxytocin (1.34±0.29 U/mg (n=10)).

Caerulein-induced acute pancreatitis in rats closely resembles interstitial-edematous pancreatitis found in clinical patients with respect to morphological, ultrastructural, biochemical and functional findings. Kinin release during the course of the experimental model is not involved in the induction of the inflammation but plays a key role in the development of the vascular symptoms, i.e. edema formation, haemoconcentration and hypovolaemia.

### EXAMPLE 9

### Xylene-Induced Cystitis

Rats were anaesthetised with pentobarbital sodium and phenobarbitone sodium as above. Following i. v. injection of Evans blue (20 mg/kg, i. v.), the urinary bladder was cannulated via the urethra using a polyethylene tubing and 250 ml xylene (30% v/v in silicone oil) were instilled within 10 s. The cannula was then left in place for a further period of 10 s. Two hours later, the animals were injected with an overdose of pentobarbitone sodium and the bladder was removed. Evans blue was extracted by incubation of the tissue in 4 ml formamide for 24 h at 55° C. for subsequent photometric measurements at 620 nm.

Experimental Cystitis

Plasma protein extravasation in the urinary bladder of anaesthetised rats was made 2 h after intravesical instillation of 30% (v/v) xylene or. Control animals were treated with appropriate volumes of the vehicles (250 ml silicone oil or 4 ml/kg 154 mmol/l NaCl solution, respectively). Oxytocin (1 mg/ml) was injected s. c. 1 h before administration of xylene or cyclophosphamide while control rats received DMSO (0.5 ml/kg, s. c.). Plasma protein extravasation is given as total tissue content of the protein marker, Evans blue, given i. v. (20 mg/kg) 5 min prior to the induction of cystitis.

The intravesical instillation of xylene (30% v/v, 250 ml) caused an acute inflammation of the urinary bladder which was quantified as the amount of plasma protein extravasation at 2 h has no significant increases in water content are present in this model. Plasma protein extravasation, quantified as the total content of the protein marker, Evans blue, was 13.21±3.68 mg which was significantly (p<0.01) higher than that of the vehicle controls (4.97±1.03 mg). Pretreatment with the oxytocin significantly reduced the effect of xylene (5.14±2.58 mg; n=10, p<0.01). Evans blue content of bladders following instillation of the vehicle (4.93±1.21 mg) was higher (p<0.01) than that in rats which had not received any intravesical instillation (2.39±0.78; n=8).

Intravesical instillations of xylene causing an acute inflammation of the urinary bladder is considered to be a model of “unspecific” cystitis which includes symptoms of increased vascular permeability and derangement of reflex and motor functions of the bladder and involves the endogenous release of kinins.

### EXAMPLE 10

### Cyclophosphamide-Induced Cystitis

Cyclophosphamide (100 mg/kg) was injected i.p. in barbiturate-anaesthetised rats for the induction of acute cystitis. Control animals were injected with an appropriate volume (4 ml/kg) of the vehicle (154 mmol/l NaCl). Five min prior to the administration of cyclophosphamide or vehicle all animals were injected i.v. with the protein marker, Evans blue (20 mg/kg). At 4 h, when increases in vascular permeability have reached maximum values in this model (Aramori I, Zenkoh J, Morikawa N, O'Donnell N. Asano M, Nakamur K et al. Novel subtype-selective nonpeptide bradykinin antagonists FR167334 and FR173657. Mol Pharmacol 1997; 51: 171-176), the uriny bladder was excised following exsanguination, and the total tissue content of Evans blue was determined as above.

Experimental Cystitis

Plasma protein extravasation in the urinary bladder of anaesthetised rats was made 4 h after i.p. injection of cyclophosphamide (100 mg/kg). Control animals were treated with appropriate volumes of the vehicles (250 ml silicone oil or 4 ml/kg 154 mmol/l NaCl solution, respectively). Oxytocin (1 mg/ml) was injected s. c. 1 h before administration of xylene or cyclophosphamide while control rats received DMSO (0.5 ml/kg, s. c.). Plasma protein extravasation is given as total tissue content of the protein marker, Evans blue, given i. v. (20 mg/kg) 5 min prior to the induction of cystitis.

The plasma protein extravasation occurring in the urinary bladder during acute cystitis induced by cyclophosphamide (100 mg/kg) was determined 4 h after the i. p. injection. While total Evans blue accumulation in the bladder following injection of 154 mmol/l NaCl was 1.25±0.47 mg (n=8), the value was increased several-fold in cyclophosphamide-treated rats (9.76±4.12 mg; n=10, p<0.01). Pre-treatment oxytocin significantly reduced plasma protein extravasation in the bladder as Evans blue accumulation was similar in rats treated with cyclophosphamide (2.56±1.28; n=8) or its vehicle (2.11±1.37; n=8).

The treatment of cancer patients with the cytostatic drug, cyclophosphamide, is associated with the development of a severe, haemorrhagic cystitis in a high proportion (up to 78-93%) of patients which can be mimicked experimentally by i. p. injection of cyclophosphamide in rats.

### EXAMPLE 11

### Collagenase-Induced Cutaneous Inflammation

One hour after anaesthesia of guinea-pigs with urethane (1.6 g/kg, i.p.), Evans blue (20 mg/kg) was injected into a jugular vein. Collagenase (10, 30 or 100 mg) or its solvent (50 ml 154 mmol/l NaCl solution) was injected s. c. into the dorsal skin. All doses of collagenase as well as the solvent were injected on both sides of the spine in randomised order. The ensuing plasma protein extravasation was determined at 60 min by extraction of Evans blue (see above) from the dorsal skin.

Treatments

Oxytocin was dissolved and diluted in DMSO for all experiments involving s. c. injection in volumes of 1.0 ml/kg. All pretreatments were given 1 h prior to the induction of the respective model of inflammation. All animal experiments approved of by Karolinska institutets animal committe.

Oxytocin was a gift from Poly Peptides Copenhagen, Denmark and was dissolved in phosphate-buffered saline (composition in mmol/l: NaCl 136.9, KCl 2.7, KH₂PO₄ 1.5, Na₂HPO₄ 7.7). Cyclophosphamide monohydrate and collagenase from Clostridium histolyticum (type II, clostridiopeptidase A, EC 3.4.24.3) were obtained from Sigma and were dissolved in 154 mmol/l NaCl.

Collagenase-induced cutaneous plasma protein extravasation. The s.c. injection of collagenase from Clostridium histolyticum into the dorsal skin of anaesthetised guinea-pigs caused a dose-dependent increase in vascular permeability. The extravasation of plasma proteins, quantified as accumulation of the protein marker, Evans blue, was increased at doses of 10-100 mg collagenase. Lower doses of collagenase did not have an effect that could be distinguished from that of the vehicle, 154 mmol/l NaCl solution (100 ml). The s.c. pre-treatment with 1 mg/ml oxytocin significantly reduced the effects of 10 mg collagenase (3.5±1.0 mg Evans blue; n=8) to (2.2±0.9 mg Evans blue; n=8) and attenuated the effect of the higher doses of the enzyme. A dose of 1 ug/ml of oxytocin had no effect on 10 and 30 mg collagenase.

Type II collagenase from Clostridium histolyticum was used to elicit increases in cutaneous vascular permeability as a model for the possible mechanism of invasion of bacteria causing life-threatening myonecrosis or gas gangrene. Plasma protein extravasation induced by collagenase in the skin of rats is only partly (about 50%) due to endogenously released kinins acting on B2 receptors while the remaining part of the response is due to 5-hydroxytryptamine release.

### EXAMPLE 12

### Topical Application of Oxytocin on Allergic Rhinitis

Six-week-old male Wistar rats were housed in an air-conditioned room maintained at about 25° C. with humidity of about 50%. Rats were given a standard laboratory rodent chow and water ad libitum.

Rats were generally sensitised by injection of 0.6 ml physiological saline containing egg albumin (1 mg), alum (2 mg) and 10/10 killed Bordetella pertussis into the four footpads on the first day. Five days later they were boosted by subcutaneous injection of 1 ml of physiological saline containing egg albumin (0.5 mg) in 10 sites on the back. Then, local sensitisation was performed every day for about one week from day 14 by dripping the egg albumin dissolved in physiological saline (1 mg/ml, 10 μl) into the bilateral nasal cavities using a micropipette.

After dripping 10 μl of egg albumin dissolved in physiological saline solution (1 mg/ml) into the bilateral nasal cavities, the numbers of nasal rubbing and sneezing were counted for 30 min and was set to 100%.

In this study, the rats were used following intranasal sensitisation (from day 21 to day 23 after the first sensitisation). On the last day, drugs (saline or oxytocin) were given topically (10 μl/each nostril) 1 h before topical antigen challenge. Nasal rubbing and sneezing was observed for 30 min after antigen challenge. Oxytocin pre-treatment caused a significant inhibition of sneezing and nasal rubbing after antigen challenge in sensitised rats. Also saline caused a minor decrease.

| | Sneezing | Nasal rubbing |
|---|---|---|
| Placebo (saline) | 93% | 86% |
| Oxytocin | 52% | 42% |

% as compared to untreated

In summary, nasal symptoms induced by antigen-antibody reaction in rats are inhibited by oxytocin.

### EXAMPLE 13

### Topical Oxytocin Prevents and Inhibits Atopic Dermatitis-Like Symptoms in Hypomagnesaemic Hairless Rats

The magnesium deficiency-induced dermatosis in hairless rats is a useful animal model for the exploration of potential treatments for cutaneous inflammatory disorders. The dermatosis develops after several days on a diet low in magnesium and is characterized by a transient erythematous maculopapular rash that remains stable for approximately 5 days. Afterwards, the signs fade even if a magnesium-deficient diet continues to be fed. Several investigators have reported that the rats appear to suffer from severe pruritus during the rash because they scratch and bite themselves causing excoriations and wounds on the trunk.

The clinical features of the erythematous pruritic rash closely mimic the acute signs of atopic dermatitis. Severe pruritus, erythematous papules associated with excoriations, vesiculation, crusting and serous exudate are the principal features of acute episodes of atopic dermatitis. It is unclear whether there are common pathogenetic mechanisms in magnesium deficiency-induced erythematous pruritic rash in rats and atopic dermatitis. However, the results obtained with drugs in this model might be of predictive value for the treatment of atopic dermatitis or other inflammatory and pruitic human skin conditions.

In the present study in rats, we have evaluated the efficacy of topically applied oxytocin.

Materials and Methods

Laboratory Animals and Maintenance Conditions

Male hairless rats (Ico:OFA hr/hr) were obtained from Iffa Credo (Lyon, France) immediately after weaning and housed under conventional standardised conditions in polycarbonate cages with wood chip bedding in a room with 12:12 h light/dark cycle, 24±1° C. The rats were fed with a standard maintenance diet low in magnesium (Altromin, Lage, Germany, C10350; 0.012% Mg²⁺/kg dry matter) and demonised water was supplied ad libitum. This diet rendered the animals hypomagnesaemic. Magnesium (mean±SD, mmol/l) in serum fell to 0.58±0.23 on day 4, 0.36±0.18 on day 10, and to the lowest value (0.24±0.11) on day 15 on the diet. The lowest concentration was maintained up to the termination of the experiments. Under these conditions, the onset of clinical signs was after 6-8 days on the diet. The first signs were pronounced reddening and swelling of both ear lobes in all animals. Most animals also showed an intense erythema on the caudal back that was present in all animals 1 day later. Simultaneously with the onset of erythema, the rats began to scratch intensively and bite themselves, causing erosions and excoriations. Head, trunk, paws and tail were uniformly and intensely reddened within 2 days after the onset of signs. This stage was followed by the development of small papules and irregularly shaped plaques 1-2 cm in diameter on the trunk. The skin lesions and the signs of pruritus were most pronounced after 10-11 days on the diet, and were then constant for approximately 4 days. This acute stage was then followed by a decrease in general erythema and, subsequently, by the remission of papules and plaques within approximately 5 days. However, complete clearance was not observed.

Drug, formulations and treatment regimens: For topical administration, the oxytocin was dissolved in ethanol/propylene glycol (1 mg/ml).

The ear lobes were chosen for topical treatment with oxytocin because they showed the greatest uniformity in inflammatory changes. The treatment was performed either unilaterally with the active drug on the right ear and with the vehicle contralaterally or bilaterally on both ears with oxytocin and vehicle, respectively. Therapeutic treatment was started immediately after clinical signs had fully developed and was given twice daily for 3 consecutive days. Prophylactic application was started on day 3 on the diet (i.e. 3-5 days before the expected onset of signs) and was given once daily for 7 days until day 9. Active drug or vehicle was applied in volumes of 20 μl to both sides of the ears.

Clinical Assessment of Activity

For assessment of the therapeutic efficacy of oxytocin the animals were examined clinically once daily during the treatment period and for a further 7 days (topical treatment). Prophylactic activity was determined by daily examinations performed during the treatment period and a further 9 days. A score index (scale 0-3) for ear lesions was applied: 0, no change; 1, slight edematous, erythematous swelling; 2, moderate edematous, erythematous swelling; 3, pronounced edematous, erythematous swelling. In all experiments, the skin lesions were evaluated clinically by a single investigator unaware of the treatment allocation.

Results

Therapeutic treatment with oxytocin applied at epicutaneously on one ear significantly reduced local erythematous swelling within 1 day after the start of treatment. Local erythematous swelling was also markedly inhibited at the vehicle-treated ear 2 days after the first dose. This was almost certainly due to contamination of the contralateral vehicle-treated ear with oxytocin by grooming. Further studies on topical activity were thus performed with bilateral drug application regimens. The local erythematous swelling of the oxytocin-treated ears was significantly inhibited within 1 day after the first dose. Signs remained suppressed until 3 days after the last dose and reappeared 4 or 5 days after the last dose. In contrast, no changes were observed on vehicle-treated ears within the treatment period.

Topical oxytocin was also efficacious when given prophylactically. The onset and the intensity of auricular inflammation were markedly suppressed on the ears of three rats treated topically with oxytocin. The ears treated with the active drug developed only a slight erythematous swelling from day 7 until day 11, whereas the vehicle-treated ears developed severe signs (sum of daily scores from day 3 to 10: 2.4±1.1 vs. 9.2±0.7). In all topical studies the appearance and course of distant erythematous lesions on the trunk and pruritus did not differ from those of rats without treatment. This indicates that the dose of oxytocin administered, which was applied on the ears, had no systemic effect.

### EXAMPLE 14

### Effect of Oxytocin on the Production of TNF-Alpha in Eosinophilic Air-Way Inflammation in Rats

Cytokines have been suggested to play key roles in the pathogenesis of bronchial asthma, which is an inflammatory disease dominated by eosinophilic granulocytes. The SDX model is a well-characterised model, primarily one of eosinophilic inflammation. Previously, Finsnes and colleagues have shown that there is a three- to fourfold increase in total BALF cell counts 24 h after SDX provocation. After 3 h, a transient increase in neutrophils occurs (Finsnes F, Christensen G, Lyberg T, Sejerstedt O M, Skjønsberg OH. Increased synthesis and release of endothelin-1 during the initial phase of airway inflammation. Am J Respir Crit Care Med 1998, 158: 1600-1606). The eosinophilic inflammation is initiated 3-6 h after intratracheal SDX provocation and lasts for >2 weeks (Finsnes F, Skjønsberg OH, Tønnessen T, Naess O, Lyberg T, Christensen G. Endothelin production and effects of endothelin antagonism during experimental airway inflammation. Am J Respir Crit Care Med 1997, 155: 1404-1412). The kinetics of this BALF finding resembles the cellular profile in human asthma, which likewise is characterised by an eosinophilic inflammatory response preceded by a transient increase in neutrophils after local allergen challenge. In addition, intravenous SDX injection increases airway hyperreactivity (Matsubara S, Fushimi K, Kikkawa H, Naito K, Ikezawa K. Difference in inhibitory effects of dexamethasone and cyclosporin A on Sephadex bead-induced airway hyperresponsiveness and inflammation in rats. Jpn J Pharmacol 1998, 77: 89-98), which is another typical feature of asthma, and this airway hyperreactivity may be inhibited by glucocorticosteroids (Matsubara et al., supra).

Experimental Procedure

12 male Wistar rats aged 11-12 weeks, with an average weight of 323 g, were used in the study. The animals (n=3 in each group) were evaluated at 3 h and 24 h after induced inflammation. These were compared with control animals (n=3 in each group) examined at the same time points. An eosinophilic airway inflammation was induced by intratracheal instillation of SDX particles (G-200 Superfine, Pharmacia & Upjohn, Uppsala, Sweden) dissolved in phosphate-buffered saline (PBS) as previously described (Andersson S E, Zackrisson C, Hemsen A, Lundberg J M. Regulation of lung endothelin content by the glucocorticosteroid budesonide. Biochem Biophys Res Commun 1992, 188:1116-1121) and compared with that in control animals receiving a similar volume of PBS intratracheally. BAL was performed by instillation of 3+2+2 ml of PBS in the right stem bronchus distal to the upper lobe, and the procedure was repeated in the main bronchus on the left side. Immediately after the BAL procedure, the lungs were removed and frozen in liquid nitrogen for tissue analyses 3 h and 24 h after SDX instillation (n=3 animals at each time point) and 3 h and 24 after intratracheal instillation of saline into control animals (n=3). Oxytocin 1 mg/ml was given s.c.

TNF-alpha was measured with a rat-specific sandwich enzyme-linked immunosorbent assay (ELISA; Factor-Test-X, Genzyme, Cambridge, Mass.). Obtained values below the detection limit of this assay system (10 pg/ml) were consequently set to 10 pg/ml. All measurements were performed in duplicate, and values are given as means.

Results

TNF-alpha had increased significantly 3 h postchallenge. Treatment with the oxytocin effectively inhibited the increase in the TNF-alpha concentration in BALF at 3 h after SDX provocation. The inhibition was most pronounced at 24 h when the TNF-alpha level in the oxytocin-treated rats was 32% of that in the animals not receiving oxytocin.

### EXAMPLE 15

### The Effect of Inhaled Oxytocin on OVA-Induced N-acetyl-LTE4 Synthesis in BN Rats—an Experimental Asthma Model

One of the most effective anti-inflammatory treatments available for asthma is glucocorticoid therapy. This is likely to be due to multiple effects on the inflammatory response, including reduced production of cytokines by lymphocytes (Barnes et al., supra) and reduced expression of adhesion molecules, such as intercellular adhesion molecule-1, by vascular endothelial cells and airway cells.

Animals

Inbred male BN rats, 7-9 weeks old, were obtained from Harlan Sprague-Dawley. Active sensitisation was performed by subcutaneous injection of 1 ml of saline containing 1 mg of ovalbumin and 3.48 mg of aluminum hydroxide. At the same time, 0.3 ml of Bordetella pertussis vaccine containing heat-killed organisms was given intraperitoneally as an adjuvant. Animals were studied 14-21 days after sensitisation.

Two groups of rats (n=3 for each group) were lightly anaesthetised with xylazine (7 mg/kg) and pentobarbital (30 mg/kg ip) before endotracheal intubation. Each rat then received either saline or 1.0 mg/kg oxytocin (1.0 mg/ml) by aerosol over a period of 5 min using a disposable nebulizer at a flow rate of 8/min and an output of 0.15 ml/min. Seventeen hours later, the animals were again anaesthetised, and these treatments were repeated. After a further hour, each of the rats was challenged with aerosolised 5% OVA in saline for 5 min.

Before the second treatment with oxytocin or saline, the rats were anaesthetised with urethane (1 g/kg ip, 50% wt/vol). After blind orotracheal intubation (6 cm of PE-240 polyethylene catheter), the common bile duct was exposed and cannulated (15 cm of PE-20 polyethylene tubing) after ligation of duodenal end. The rats were allowed to stabilise for a period of 2 h before challenge with OVA. Bile was collected for 1 h before and for 2 consecutive periods of 1 h after OVA challenge (0-1 and 5-6). All bile samples were collected on ice in 1.5-ml Eppendorf tubes. The bile was kept frozen at 80° C. before analysis.

Bile samples were thawed, and methanol was added to 0.3-ml aliquots to give a final concentration of 80%. After centrifugation, the supernatants were adjusted to a concentration of 30% methanol and a pH of 3 and subjected to precolumn extraction reverse-phase HPLC. The mobile phase consisted of a mixture of 64% methanol in aqueous buffer (1 mM EDTA and 0.1% acetic acid, adjusted to a pH of 5.4 by the addition of ammonium hydroxide). The flow rate was 0.7 ml/min. Ultraviolet absorbance was monitored by a variable wavelength ultraviolet detector. The retention time of standard N-acetyl-LTE4 using these conditions was about 20 min.

Radioimmunoassay

Column fractions were evaporated to dryness in a centrifuge under vacuum, and the residues were dissolved in phosphate-buffered saline (0.1 ml at pH 8.2). N-acetyl-LTE4 was measured in each fraction by using a monoclonal antibody directed against LTC4, which cross-reacted with N-acetyl-LTE4. [14,15-³H]LTC4 was used as the radioactive ligand.

Results

Rats were treated with either oxytocin or saline by aerosol. N-acetyl-LTE4 were measured before and at various time intervals after ovalbumin (OVA) challenge. The biliary levels of N-acetyl-LTE4 before OVA challenge and during periods corresponding to the early response (0-1 h after challenge) and the late response (5-6 h after challenge). The levels of N-acetyl-LTE4 before challenge were almost identical in the oxytocin-treated and control groups. Pretreatment with oxytocin had a small inhibitory effect on the excretion of N-acetyl-LTE4 during the early response (4.3±2.1 vs. 6.9±1.8 pmol/h). In contrast, oxytocin eliminated the increase in the levels of N-acetyl-LTE4 observed in the control rats during the late response (2.6±0.8 vs. 5.2±0.9 pmol/h). For comparison, the basal level of N-acetyl-LTE4 in bile before antigen challenge was 2.2±0.4 pmol/h.

1. A method for treating a subject suffering from or having an inflammation condition, comprising administering to said subject a substance with oxytocin activity.

2. The method according to claim 1, characterised in that the inflammation is selected from the group consisting of edema, hyperalgesia, myeloperoxidase accumulation, cystitis, pancreatitis, cutaneous inflammation, allergic rhinitis, dermatitis, air-way inflammation, and asthma.

3. The method according to claim 1, characterised in that the substance is selected from the group consisting of the following compounds:

| | |
|---|---|
| X₉ S | |
| X₁-X₂-X₃-X₄-Asn-Cys-X₅-X₆-X₇-X₈-NH₂ | (SEQ ID NO: 2) |

wherein
X₁ is selected from the group consisting of Cys, Mpa and nothing,
X₂ selected from the group consisting of Tyr, (O-methyl-Tyr), Phe, and nothing,
X₃ is selected from the group consisting of Ile, Val, Hoph, Phe, Cha, and nothing,
X₄ is selected from the group consisting of Gln, Ser, Thr, Cit, Arg, and Daba,
X₅ is selected from the group consisting of Pro, and nothing,
X₆ is selected from the group consisting of Ile, Leu, nothing, Val, Hos, Daba, Thr, Arg, and Cit,
X₇ is selected from the group consisting Gly, nothing, and Ala,
X₈ is selected from the group consisting Gly, and nothing,
X₉ is selected from the group consisting CH₂ and S;
as well as salts thereof.

4. The method according to claim 1, characterised in that the substance is selected from the group consisting of the following compounds: SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, and SEQ ID NO: 24.

5. The method according to claim 1, characterised that the pharmaceutical composition comprises substances that increase the release of oxytocin and/or the number or affinity of oxytocin receptors, such as oestrogen, or drugs having an α₂-agonistic effect, such as clonidine.

6. The method according to claim 1, characterised in that the substance is administered in amount of 0.01-100 ng/kg body weight of the patient.

7. The method according to claim 1, characterised in that the substance is administered in amount of 0.1-10 ng/kg body weight of the patient.

8. Pharmaceutical composition against inflammation, characterised in that it comprises an effective concentration of at least one substance with oxytocin activity in mixture or otherwise together with at least one pharmaceutically acceptable carrier or excipient, wherein the substance is selected from the group consisting of the following compounds:

| | |
|---|---|
| X₉ S | |
| X₁-X₂-X₃-X₄-Asn-Cys-X₅-X₆-X₇-X₈-NH₂ | (SEQ ID NO: 2) |

wherein
X₁ is selected from the group consisting of Cys, Mpa and nothing,
X₂ is selected from the group consisting of Tyr, (O-methyl-Tyr), Phe, and nothing,
X₃ is selected from the group consisting of Ile, Val, Hoph, Phe, Cha, and nothing,
X₄ is selected from the group consisting of Gln, Ser, Thr, Cit, Arg, and Daba,
X₅ is selected from the group consisting of Pro, and nothing,
X₆ is selected from the group consisting of Ile, Leu, nothing, Val, Hos, Daba, Thr, Arg, and Cit,
X₇ is selected from the group consisting Gly, nothing, and Ala,
X₈ is selected from the group consisting Gly, and nothing,
X₉ is selected from the group consisting CH₂ and S;
as well as salts thereof.

9. Pharmaceutical composition according to claim 8, characterised in that the inflammation is selected from the group consisting of edema, hyperalgesia, myeloperoxidase accumulation, cystitis, pancreatitis, cutaneous inflammation, allergic rhinitis, dermatitis, air-way inflammation, and asthma.

10. Pharmaceutical composition according to claim 8, characterised in that the substance is selected from the group consisting of the following compounds: SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, and SEQ ID NO: 24.

11. Pharmaceutical composition according to claim 8, characterised in that it comprises substances that increase the release of oxytocin and/or the number or affinity of oxytocin receptors, such as oestrogen, or drugs having an α₂-agonistic effect, such as clonidine.

12. Pharmaceutical composition according to claim 8, characterised in that the effective concentration is 4-70% by weight, preferably 0.1-50% by weight.

13. Pharmaceutical composition according to claim 9, characterised in that the substance is selected from the group consisting of the following compounds: SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, and SEQ ID NO: 24.

14. Pharmaceutical composition according to claim 9, characterised in that it comprises substances that increase the release of oxytocin and/or the number or affinity of oxytocin receptors, such as oestrogen, or drugs having an α₂-agonistic effect, such as clonidine.

15. Pharmaceutical composition according to claim 10, characterised in that it comprises substances that increase the release of oxytocin and/or the number or affinity of oxytocin receptors, such as oestrogen, or drugs having an α₂-agonistic effect, such as clonidine.

16. Pharmaceutical composition according to claim 9, characterised in that the effective concentration is 4-70% by weight, preferably 0.1-50% by weight.

17. Pharmaceutical composition according to claim 10, characterised in that the effective concentration is 4-70% by weight, preferably 0.1-50% by weight.

18. Pharmaceutical composition according to claim 11, characterised in that the effective concentration is 4-70% by weight, preferably 0.1-50% by weight.


SEQUENCE LISTING (24 sequences)

SEQ ID NO: 1 - protein, 9 aa, Artificial Sequence
  Description of Artificial Sequence Synthetic Peptide
CYIQNCPLG

SEQ ID NO: 2 - protein, 10 aa, Artificial Sequence
  Description of Artificial Sequence Synthetic Peptide
XXXXNCXXXX

SEQ ID NO: 3 - protein, 9 aa, Artificial Sequence
  Description of Artificial Sequence Synthetic Peptide
CYIQNCPIG

SEQ ID NO: 4 - protein, 9 aa, Artificial Sequence
  Description of Artificial Sequence Synthetic Peptide
CYISNCPIG

SEQ ID NO: 5 - protein, 9 aa, Artificial Sequence
  Description of Artificial Sequence Synthetic Peptide
CFVRNCPTG

SEQ ID NO: 6 - protein, 9 aa, Artificial Sequence
  Description of Artificial Sequence Synthetic Peptide
CYIQNCPRG

SEQ ID NO: 7 - protein, 9 aa, Artificial Sequence
  Description of Artificial Sequence Synthetic Peptide
CYFQNCPRG

SEQ ID NO: 8 - protein, 10 aa, Artificial Sequence
  Description of Artificial Sequence Synthetic Peptide
CYIQNCPLGG

SEQ ID NO: 9 - protein, 6 aa, Artificial Sequence
  Description of Artificial Sequence Synthetic Peptide
CYIQNC

SEQ ID NO: 10 - protein, 7 aa, Artificial Sequence
  Description of Artificial Sequence Synthetic Peptide
CYIQNCP

SEQ ID NO: 11 - protein, 8 aa, Artificial Sequence
  Description of Artificial Sequence Synthetic Peptide
CYIQNCPL

SEQ ID NO: 12 - protein, 8 aa, Artificial Sequence
  Description of Artificial Sequence Synthetic Peptide
YIQNCPLG

SEQ ID NO: 13 - protein, 7 aa, Artificial Sequence
  Description of Artificial Sequence Synthetic Peptide
IQNCPLG

SEQ ID NO: 14 - protein, 6 aa, Artificial Sequence
  Description of Artificial Sequence Synthetic Peptide
QNCPLG

SEQ ID NO: 15 - protein, 5 aa, Artificial Sequence
  Description of Artificial Sequence Synthetic Peptide
IQNCP

SEQ ID NO: 16 - protein, 9 aa, Artificial Sequence
  Description of Artificial Sequence Synthetic Peptide
CYXXNCPRG

SEQ ID NO: 17 - protein, 9 aa, Artificial Sequence
  Description of Artificial Sequence Synthetic Peptide
CYVTNCPLG

SEQ ID NO: 18 - protein, 9 aa, Artificial Sequence
  Description of Artificial Sequence Synthetic Peptide
CYXTNCPVG

SEQ ID NO: 19 - protein, 9 aa, Artificial Sequence
  Description of Artificial Sequence Synthetic Peptide
CYFXNCPLG

SEQ ID NO: 20 - protein, 9 aa, Artificial Sequence
  Description of Artificial Sequence Synthetic Peptide
CYXRNCPXA

SEQ ID NO: 21 - protein, 9 aa, Artificial Sequence
  Description of Artificial Sequence Synthetic Peptide
CYVXNCPXA

SEQ ID NO: 22 - protein, 9 aa, Artificial Sequence
  Description of Artificial Sequence Synthetic Peptide
CYXXNCPXA

SEQ ID NO: 23 - protein, 9 aa, Artificial Sequence
  Description of Artificial Sequence Synthetic Peptide
CYFRNCPVA

SEQ ID NO: 24 - protein, 9 aa, Artificial Sequence
  Description of Artificial Sequence Synthetic Peptide
XXIQNCPLG